(19) United States
(12) Patent Application Publication
(10) Pub. No.: US 2026/0354846 A1
(43) Pub. Date: Nov. 12, 2026

(54) DEVICES, SYSTEMS, AND METHODS FOR DISRUPTION AND/OR REMOVAL OF FECAL MATTER

(71) Applicant: ShockEvac Medical, Inc., Portola Valley, CA (US)

(72) Inventors: George Triadafilopoulos, Portola Valley, CA (US); Peter Garcia-Meza, San Francisco, CA (US); Patrick Wu, San Carlos, CA (US); Daniel Chan, Sunnyvale, CA (US)

(21) Appl. No.: 19/103,110

(22) PCT Filed: Aug. 11, 2023
(86) PCT No.: PCT/US23/72114

§ 371 Date: Feb. 11, 2025

Related U.S. Application Data
(60) Provisional application No. 63/371,160, filed on Aug. 11, 2022.
(60) Provisional application No. 63/387,256, filed on Dec. 13, 2022.

(51) Int. Cl. A61B 17/22; A61B 17/00

(52) U.S. Cl. CPC A61B 17/22; A61B 2017/00398; A61B 2017/22037; A61B 2017/22051; A61B 2017/22079

(57) ABSTRACT

Devices, systems, and methods for disrupting fecal matter in a body lumens are disclosed herein. According to some embodiments, the present technology includes a disruptor comprising a proximal region configured to be handled by a user, a distal region, and an elongated member extending therebetween, wherein the disruptor further comprises a distal element carried by a distal portion of the elongate member and configured for insertion into the intestinal lumen through an anus of the patient and an energy source operably coupled to the distal element. The device can include an expandable member configured to be positioned in the intestinal lumen, the expandable member having a collapsed state for introduction into the intestinal lumen and an expanded state in which the expandable member is configured to conform to the inner wall of the intestinal lumen to prevent or reduce leakage of intestinal fluids and/or fecal matter through the anus to an extracorporeal location.

### CROSS-REFERENCE TO RELATED APPLICATION(S)

The present application incorporates by reference in its entirety U.S. Provisional Patent Application No. 63/371,160, filed Aug. 11, 2022, and U.S. Provisional Patent Application No. 63/387,256, filed Dec. 13, 2022.

### TECHNICAL FIELD

The present technology relates to devices, systems, and methods for the disruption and/or removal of fecal matter.

### BACKGROUND

Obstructive constipation (OC) and fecal impaction (FI) occur because hardened fecal matter is retained in the distal large bowel and cannot be evacuated by regular peristaltic colonic activity and coordinated pelvic floor muscle contractility. If OC and FI are not recognized and treated early, they can give rise to the formation of large fecaliths or stone-like feces. OC and FI cause increased morbidity and a significant decrease in the patient's quality of life. OC and FI commonly occur among the elderly who are hospitalized, bedridden or under institutional, long-term care. Among hospitalized patients, administration of opioids can result in OC and FI, especially in those with a prior history of chronic constipation and are bedridden. OC and FI also account for frequent and repetitive visits to the emergency departments (ED), increasing health care costs because of acute medical visits, medical and nursing personnel engagement, ED overcrowding and ambulance transportation.

Severe constipation, the leading predisposing condition to FI, is a significant problem that affects almost 70% of elderly residing in nursing homes. Among those affected by FI, only 7% are detected by a digital rectal examination and further imaging studies are frequently needed. FI is more common among older women with pelvic floor descent from previous multiparity or pelvic surgery who may be institutionalized or have associated neuropsychiatric disorders and it can lead to complications, such as vaginal and urinary prolapse, recurrent urinary tract infections, morbidity, and mortality.

The continuous contact between the hard, inspissated feces and the colonic mucosa may also cause an increase in mucus secretion and overflow fecal incontinence. FI may also cause an increase in intraluminal pressure in the colon which causes a decrease in perfusion of the colonic wall. The resulting inflammation can give rise to stercoral ulcers, recurrent rectal bleeding and sometimes rectosigmoid perforation attributed to feces being hardest in the rectosigmoid colon.

Patients with OC and FI often present with a history of inability to evacuate stools spontaneously (defecatory dysfunction) and complain of total outlet constipation. These patients also report the need for straining at defecation and the need for digital manipulation, either through the rectum or the vagina or both. In most instances, there is an associated history of progressive abdominal distension with increasing abdominal discomfort or pain. Physical examination often reveals a distended abdomen. In thinly built or emaciated individuals, a hard fecal mass may be palpable along the left colon, reaching deep into the pelvis. Occasionally, patients may also present with overflow diarrhea (incontinence) and fecal soiling of underwear. The diagnosis of OC and FI is based on both a carefully elicited history and clinical signs.

A digital rectal examination is mandatory to confirm the diagnosis of OC and FI, but this is frequently uncomfortable to the patient and omitted or avoided by clinicians and nursing personnel. Regardless, in the proper clinical setting, when a rectal examination does not reveal fecal impaction or a hard fecal mass in the rectum, the possibility of fecal impaction more proximally (typically in the sigmoid colon) or other causes of obstipation, such as strictures, colonic malignancy, or colonic volvulus, should be considered. The most useful and commonly used radiological imaging for evaluation is a CT abdomen, preferably with oral or rectal contrast. A plain x-ray of the abdomen can, at times, reveal fecal overloading of the colon, with colonic distention of the segment proximal to the region of fecal impaction. Rarely, a contrast enema or sigmoidoscopy are used.

Treatment options include a digital evacuation of the impacted fecal mass or the rectal administration of stool softening agents, usually enemas or suppositories, but often manual disimpaction is required. This is an essential therapeutic procedure if one can palpate hard stool in the rectal area. The procedure is best done using ample lubrication and by gentle removal of the impacted stool using the index and middle fingers. Most patients find immediate relief once fecal disimpaction is effectively performed. Unfortunately, this is painful to the patient and time consuming to the operator, sometimes requiring general anesthesia. Further, once the evacuation occurs, the amount of soiling that follows is significant and universally requires a time-consuming cleansing, to avoid skin breakdown that-in turn-leads to decubitus ulcers and skin infections, cellulitis, and the need for extensive debridement and/or plastic surgery.

If the abdominal x-ray reveals that the fecal impaction is located distally, then the use of enemas and suppositories can be helpful. The enema is best delivered with a catheter past the hardened stool, but this is challenging to accomplish given the soft nature of the catheter that may coil. The best method is to use ample water and combine it with laxatives, such as docusate or sorbitol. It is important to administer small amounts (60-120 ml) of enema so as not to create discomfort in the patient. Once the enema is administered, one can help the process of evacuation by gently massaging the lower abdomen. This process may have to be repeated several times until only clear liquid passes and the rectal vault is checked empty upon repeat digital examination. For proximal fecal impaction, the ideal laxative is orally administered polyethylene glycol. One may have to administer 4 liters of polyethylene glycol over a period of 4-6 hours before a response is seen. If abdominal cramps and nausea occur, then no more polyethylene glycol should be ingested. Another laxative that can help with proximal impaction is magnesium citrate. Polyethylene glycol can be given orally only if conditions, like volvulus or structural bowel obstruction due to any cause, are ruled out. Administration of an appropriate laxative or fiber supplement with increased intake of water after the evacuation of the impacted fecal mass is administered to prevent recurrence. Early surgical evaluation and intervention are necessary if there are associated signs of peritonitis. Such intervention is technically very challenging and frequently requires a temporary or permanent colostomy placement. Stercoral perforation is associated with a high mortality in the elderly if not recognized and treated early. Sometimes impaction occurs after a barium study; to mitigate this risk, all patients should be encouraged to consume extra fluids after the procedure. In some cases, preventive use of an osmotic laxative, like sorbitol or milk of magnesia, may be helpful.

In people who have undergone repair of anal fistulas or surgical removal of hemorrhoids, OC and FI are very common. These are related to use of opiates, sphincter spasm, edema around the anal tissues, and a fear of going to the bathroom due to pain. In most mild cases, administration of an enema is sufficient; but in severe cases, one may need to perform manual disimpaction in the operating room. The patient will usually need some type of anesthesia to relax the anal sphincter complex before FI can be removed.

OC and FI are significant but preventable problems in the elderly population residing in hospitals and other long-term facilities. The best treatment for OC and FI is prevention. Often these patients present to the ED because of symptoms that mimic other sinister intestinal pathology, such as colorectal cancer.

OC and particularly FI are serious disorders that affect many older adults. The disorders are best managed by an interprofessional team that includes a physician, nurse, pharmacist, neurologist, social worker, and physical therapist. The pharmacist should ensure that the patient is not on any medications that slow down bowel movements and avoid polypharmacy. The dietitian should encourage an increase in fluid intake and high-fiber diet, as needed. Both exercise and ambulation are vital. But it is usually the nurse or medical assistant who must manage the patient with FI diagnosed by the physician. Education of the family and the patient about dietary habits and lifestyle are essential to preventing recurrences. If chronic constipation cannot be cured, then attempts at improving the quality of life using various laxative regimens must be made.

OC and FI can significantly affect quality of life. Despite the availability of medications, recurrences remain chronic and recurrent problems. Many of these patients are old, non-ambulatory, and reside in a nursing home. Thus, it is important to improve the quality of life rather than subject these patients to drastic medical interventions, surgery, or regular invasive procedures which also have the potential to cause iatrogenic complications. With the current opioid crisis, an increasing number of young and middle-aged adults also suffer from chronic constipation and FI that result from frequent use of narcotics and they are projected to represent an increasing percentage of the victims of these conditions.

Accordingly, there is a need for improved fecal disimpaction tools.

### SUMMARY

The present technology relates to devices, systems, and methods for the efficient disruption and/or removal of fecal matter. The present technology includes, for example, a hand-held device configured for insertion through the anus into the rectum to engage impacted fecal matter. The device can include a disruption element that, when activated, translates, rotates, and/or vibrates at the treatment site to dislodge or otherwise disrupt impacted fecal matter. The disruption element can be coupled to a hub that is configured to be positioned within the anal canal to help support and anchor the disruption element during use, and to prevent intestinal fluids and/or fecal matter from leaking out of the rectum through the anus. In some embodiments, the device includes an infusion channel for the delivery of saline, laxatives, and/or other fluids into the rectum to soften the impacted stool. In these and other embodiments, the device can include an aspiration channel that evacuates the dislodged and/or softened fecal matter and other fluids into a vacuum canister.

The subject technology is illustrated, for example, according to various aspects described below, including with reference to FIGS. 1A-21. Various examples of aspects of the subject technology are described as numbered clauses (1, 2, 3, etc.) for convenience. These are provided as examples and do not limit the subject technology.
1. A device for removing fecal matter from an intestine of a patient, the device comprising:
a proximal portion configured to be engaged by a user, and a distal portion, configured to be positioned at a treatment site within a lumen of the intestine proximate to impacted fecal matter;
a disruptor comprising a proximal region configured to be handled by a user, a distal region, and an elongated member extending therebetween, wherein the disruptor further comprises:
   a distal element carried by a distal portion of the elongate member and configured for insertion into the intestinal lumen through an anus of the patient,
   an energy source operably coupled to the distal element, and
   wherein activation of the energy source energizes the distal element, thereby causing the distal element to deliver energy to the impacted fecal matter and dislodge a portion of the impacted fecal matter;
an expandable member configured to be positioned in the intestinal lumen, the expandable member having a collapsed state for introduction into the intestinal lumen and an expanded state in which the expandable member is configured to conform to the inner wall of the intestinal lumen to prevent or reduce leakage of intestinal fluids and/or fecal matter through the anus to an extracorporeal location; and
a channel extending distally from a port at the proximal portion of the device to an opening at the distal portion of the device, wherein the port is configured to be fluidly coupled to a negative pressure source to apply a negative pressure at the treatment site to remove the portion of the fecal matter and/or other matter present in the intestinal lumen.
2. The device of Clause 1, wherein the channel is a first channel, the port is a first port, and the opening is a first opening, and wherein the device further comprises a second channel extending distally from a second port at the proximal portion of the device to a second opening at the distal portion of the device, wherein the second port is configured to be fluidly coupled to a fluid source to deliver a fluid through the second channel to the treatment site.
3. The device of any one of the previous Clauses, wherein the channel is a first channel, the port is a first port, and the opening is a first opening, and wherein the device further comprises a third channel extending distally from a third port at the proximal portion of the device to a third opening at the expandable member, wherein the third port is configured to be fluidly coupled to a positive pressure source to transform the expandable member from a collapsed state to an expanded state.
4. The device of any one of the previous Clauses, wherein the expandable member is a balloon.
5. The device of any one of the previous Clauses, wherein the distal portion is configured for insertion into the colon in an uncovered state.
6. The device of any one of the previous Clauses, wherein the device has a collapsed state for delivery through the anus, and wherein the device in the collapsed state has a cross-sectional dimension of at least 0.5 inches.
7. The device of any one of the previous Clauses, further comprising a proximal element configured to be positioned proximal of the anus at an extracorporeal location, wherein the proximal element has a diameter that is greater than a diameter of the anus such that the expandable member and the proximal element together secure the device at the anus.
8. The device of any one of the previous Clauses, wherein the energy source is a motor, and wherein activation of the motor causes the distal element to move, thereby dislodging a portion of the fecal matter.
9. The device of Clause 8, wherein activation of the motor causes the distal element to vibrate, translate, and/or rotate.
10. The device of any one of the previous Clauses, wherein the energy source is configured to deliver high frequency ultrasound to the impacted fecal matter via the distal element.
11. The device of any one of the previous Clauses, further comprising a hub, wherein the expandable member is a first portion of a hub and the hub further comprises a second, non-expandable portion.
12. The device of Clause 11, wherein the first portion of the hub is configured to be positioned in a rectum of the patient and the second portion of the hub is configured to be positioned within an anal canal of the patient.
13. A device for removing fecal matter from an intestine of a patient, the device comprising:
a proximal portion configured to be engaged by a user and a distal portion configured to be positioned at a treatment site within a lumen of the intestine proximate to impacted fecal matter;
a disruptor comprising a proximal region configured to be handled by a user, a distal region, and an elongate member extending therebetween, wherein the disruptor further comprises a distal element carried by a distal portion of the elongate member and configured for insertion into the intestinal lumen through an anus of the patient, wherein the distal element is actuatable to dislodge a portion of the impacted fecal matter; and
a hub having a distal portion configured to be positioned in the intestinal lumen, a proximal portion configured to be positioned proximal of the anus at an extracorporeal location, and an intermediate portion extending between the distal and proximal portions of the hub that is configured to be positioned along the anal canal, wherein the hub further comprises a channel that is configured to slidably receive the elongate member of the disruptor therethrough.
14. The device of Clause 13, wherein the distal portion of the hub comprises an expandable member having a collapsed state for introduction into the intestinal lumen and an expanded state in which the expandable member is configured to conform to the inner wall of the intestinal lumen to prevent or reduce leakage of intestinal fluids and/or fecal matter through the anus to an extracorporeal location.
15. The device of Clause 14, wherein the expandable member is configured to be deployed in the rectum, adjacent a distal end of the anal canal.
16. The device of Clause 14 or Clause 15, wherein the proximal portion of the hub has an axial cross-sectional dimension greater than a diameter of the anus such that the proximal portion of the hub cannot move distally through the anus.
17. The device of Clause 16, wherein the proximal portion of the hub has a substantially fixed cross-sectional dimension.
18. The device of Clause 16, wherein the proximal portion of the hub has a collapsed state and an expanded state in which the hub has the axial cross-sectional dimension greater than the diameter of the anus.
19. The device of any one of the previous Clauses, wherein the channel is a first channel and the hub further comprises a second channel extending distally from a proximal opening at the proximal portion of the hub to a distal opening at the distal portion of the hub, wherein the second channel is configured to be fluidly coupled to a negative pressure source to apply a negative pressure at the treatment site to remove the portion of the fecal matter and/or other matter present in the intestinal lumen.
20. The device of Clause 19, wherein the second channel is configured to be fluidly coupled to a fluid source and deliver a fluid to the treatment site.
21. The device of Clause 19, wherein the hub further comprises a third channel extending distally from a proximal opening at the proximal portion of the hub to a distal opening at the distal portion of the hub, wherein the third channel is configured to be fluidly coupled to a fluid source and deliver a fluid to the treatment site.
22. The device of any one of the previous Clauses, wherein the hub comprises a sidewall defining an interior region of the hub that is configured to receive all or a portion of the distal element therein.
23. The device of any one of the previous Clauses, wherein the hub has a collapsed configuration and an expanded configuration, and wherein, at least in the collapsed configuration, the hub has a tapered distal region.
24. A method for removing fecal matter from an intestine of a patient, the method comprising:
positioning a treatment device through the anus to a treatment site within an intestine of a patient proximate to impacted fecal matter, wherein the treatment device comprises (a) a hub and (b) a disruptor including an elongated member and a distal element carried by a distal portion of the elongated member, the disruptor configured to be operably coupled to an energy source, and wherein the treatment device is positioned such that the hub is positioned on either side of the anus;
expanding a portion of the hub that is positioned within the rectum such that the portion conforms to an inner surface of the rectal lumen;
engaging the impacted fecal matter with the distal element of the disruptor and activating an energy source of the disruptor to deliver energy to the treatment site via the distal element, thereby dislodging a portion of the impacted fecal matter; and
aspirating the dislodged portion of the impacted fecal matter through a channel in the hub.
25. The method of Clause 24, wherein the channel is a first channel and the hub comprises a second channel configured to receive the elongated member of the disruptor therethrough, and wherein the method further comprises translating the elongated member within the second channel to advance and retract the distal element of the disruptor.
26. The method of Clause 24 or Clause 25, wherein the channel is a first channel and the hub comprises a third channel extending through at least a portion of the length of the hub, and wherein the method further comprises delivering a fluid to the treatment site through the third channel.
27. The method of any one of the previous Clauses, wherein engaging the impacted fecal matter includes rotation and/or translation of the distal element of the disruptor.
28. The method of any one of the previous Clauses, wherein engaging the impacted fecal matter includes delivering ultrasound energy to the fecal matter.
29. The method of any one of the previous Clauses, wherein the portion of the hub is a first portion of the hub, and wherein positioning the treatment device includes positioning a second portion of the hub along the anal canal.
30. The method of any one of the previous Clauses, wherein the portion of the hub is a first portion of the hub, and wherein positioning the treatment device includes positioning a third portion of the hub proximal of and up against the anus.
31. The method of any one of the previous Clauses, wherein positioning the treatment device includes inserting the hub through the anus with a distal region of the distal element extending distally from a distal end of the hub.
32. The method of any one of the previous Clauses, wherein the energy source of the disruptor is a motor.
33. A method of removing fecal matter from the intestine of a patient, comprising:
introducing into the rectum a device configured to aid insertion of a mechanical energy source;
securing the device to abut and seal itself against the inner surface of the anal canal and stabilize itself in the anus;
introducing liquids through the device to soften fecal matter; and
activating said device mechanically rotates a brush to break down fecal matter into small pieces to allow passive and/or active evacuation of liquified fecal matter.
34. The method according to Clause 33, wherein inflating a distal balloon abuts itself against the inner surface of the anal canal to achieve a seal and prevent leaking.
35. The method according to Clause 33, wherein a sealing proximal feature stabilizes the device in the rectum.
36. The method according to Clause 33, wherein the device allows injection of liquids into the rectum to soften fecal matter and evacuation of liquefied fecal matter from the rectum.
37. The method according to Clause 33, wherein the mechanical energy source comprises a brush and a drive module, and wherein the brush is attached to the distal end of the drive module, and wherein the drive module is configured to oscillate, rotate, or advance the brush.
38. The method according to Clause 33, wherein activating the mechanical energy provides a rotational motion to break down fecal matter.
39. The method according to Clause 33, wherein the rotational direction can be clockwise or counter clockwise.
40. The method according to Clause 33, wherein advancing and retracting the rotating brush breaks down fecal matter.
41. The method according to Clause 33, wherein moving the rotational brush in a non-axial matter relative to the device anchored in the rectum breaks down fecal matter.
42. The method according to Clause 33, wherein the device allows passive evacuation of liquified fecal matter by gravity.
43. The method according to Clause 33, wherein the device allows active evacuation of liquified fecal matter by vacuum.
44. The method according to Clause 33, wherein the device allows active flushing of the evacuation port to clear fecal matter lodged in evacuation tubing.
45. The method according to Clause 33, wherein the evacuation tubing is connected to a collection canister.
46. A device for removing fecal matter from an intestine of a patient, the device comprising:
a proximal portion configured to be engaged by a user, and a distal portion, configured to be positioned at a treatment site within a lumen of the intestine proximate to impacted fecal matter;
a disruptor comprising a proximal region configured to be handled by a user, a distal region, and an elongated member extending therebetween, wherein the disruptor further comprises:
   a distal element carried by a distal portion of the elongate member and configured for insertion into the intestinal lumen through an anus of the patient,
   an energy delivery element operably coupled to the distal element, and
   wherein activation of an energy source causes energy delivery element to direct energy towards the impacted fecal matter and dislodge a portion of the impacted fecal matter;
an expandable member configured to be positioned in the intestinal lumen, the expandable member having a collapsed state for introduction into the intestinal lumen and an expanded state in which the expandable member is configured to conform to the inner wall of the intestinal lumen to prevent or reduce leakage of intestinal fluids and/or fecal matter through the anus to an extracorporeal location; and
a channel extending distally from a port at the proximal portion of the device to an opening at the distal portion of the device, wherein the port is configured to be fluidly coupled to a negative pressure source to apply a negative pressure at the treatment site to remove the portion of the fecal matter and/or other matter present in the intestinal lumen.
47. The device of Clause 46, wherein the energy delivery element is a reflector configured to reflect ultrasonic energy at the treatment site.
48. The device of Clause 46, wherein the energy delivery element is a reflector configured to reflect ultrasonic energy at the treatment site, and wherein the ultrasonic energy is generated by the energy source that is positioned at an extracorporeal location.
49. A device for removing fecal matter from an intestine of a patient, the device comprising:
a proximal portion comprising a handle configured to be engaged by a user and a distal portion configured to be positioned at a treatment site within a lumen of the intestine proximate to impacted fecal matter, the device having a longitudinal axis extending between the proximal and distal portions;
an elongate member extending between a proximal region at the handle and a distal region;
an elongate distal element at the distal portion of the device, wherein the distal element is configured for insertion into the intestinal lumen through an anus of the patient; and
a hub positioned between the distal region of the elongate member and the distal element along the longitudinal axis of the device, the hub having a distal portion configured to be positioned in the intestinal lumen, a proximal portion configured to be positioned proximal of the anus at an extracorporeal location, and an intermediate portion extending between the distal and proximal portions of the hub and configured to be positioned along the anal canal.
50. The device of Clause 49, wherein the elongate member comprises a bend along its length, wherein the bend positions a longitudinal axis of the distal element at an angle relative to the longitudinal axis of the handle.
51. The device of Clause 49, wherein the elongate member comprises a bend along its length, wherein the bend positions a longitudinal axis of the hub at an angle relative to the longitudinal axis of the handle.
52. The device of Clause 48 or Clause 49, wherein an outer surface of the distal element comprises one or more openings configured to deliver a fluid from an extracorporeal fluid source to the treatment site.
53. The device of any one of Clauses 48 to 52, wherein the distal element is actuatable to dislodge a portion of the fecal matter.
54. The device of any one of Clauses 48 to 53, wherein the distal element and the hub are detachable from the elongate member.
55. The device of any one of Clauses 48 to 54, wherein the distal element, hub, and elongate member are detachable from the handle.
56. The device of any one of Clauses 48 to 55, wherein the distal element and hub are disposable.
57. The device of any one of Clauses 48 to 56, wherein the handle is reusable.
58. The device of any one of Clauses 48 to 57, wherein the elongate member is reusable.
59. The device of any one of Clauses 48 to 58, further comprising an energy source operably coupled to the distal element, wherein activation of the energy source energizes the distal element, thereby causing the distal element to deliver energy to the fecal matter and dislodge a portion of the impacted fecal matter.
60. The device of Clause 59, wherein the energy source is a motor, and wherein activation of the motor causes the distal element to move, thereby dislodging a portion of the fecal matter and/or stimulate muscles adjacent the rectum.
61. The device of Clause 60, wherein activation of the motor causes the distal element to vibrate.
62. The device of Clause 60 or Clause 61, wherein activation of the motor causes the distal element to swirl about its proximal end such that a longitudinal axis of the distal element is angled relative to a longitudinal axis of the hub during swirling of the distal element.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A and 1B depict a treatment device in accordance with the present technology, shown in different configurations.

FIG. 2 is a side cross-sectional view of the hub of the treatment device of FIGS. 1A and 1B in accordance with several embodiments of the present technology. In FIG. 2, the hub is in an expanded configuration.

FIGS. 3A-3D depict a method of using the treatment device of FIGS. 1A and 1B to treat fecal impaction in accordance with several embodiments of the present technology.

FIGS. 4, 5, 6, 7, 8, 9, 10, 11, 12, 13, 14, 15 and 16 show various distal elements configured for use with the disruptors of the treatment devices of the present technology.

FIGS. 17, 18 and 19 show various hubs configured for use with the treatment devices of the present technology.

FIG. 20 is an example control diagram for the treatment devices of the present technology.

FIG. 21 depicts a treatment device configured in accordance with the present technology, shown with a distal portion positioned in the rectum.

### DETAILED DESCRIPTION

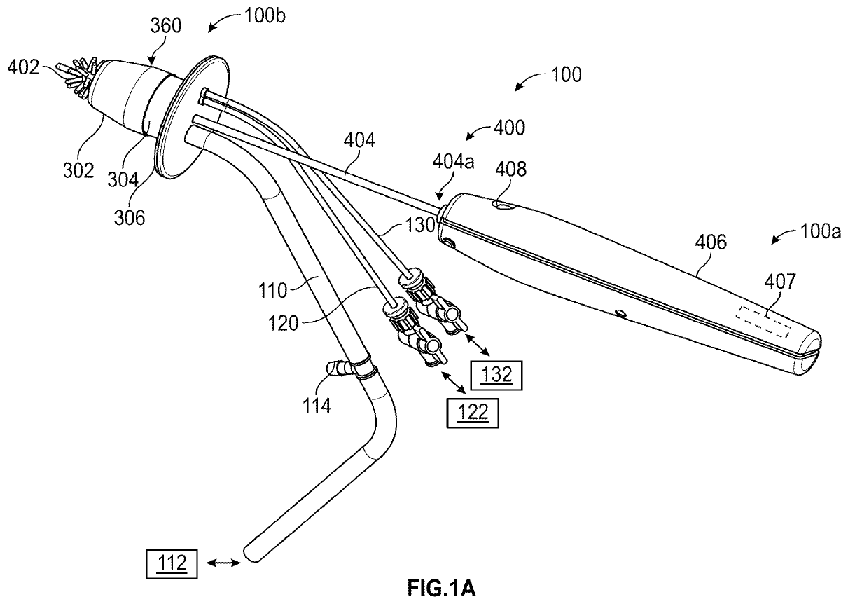

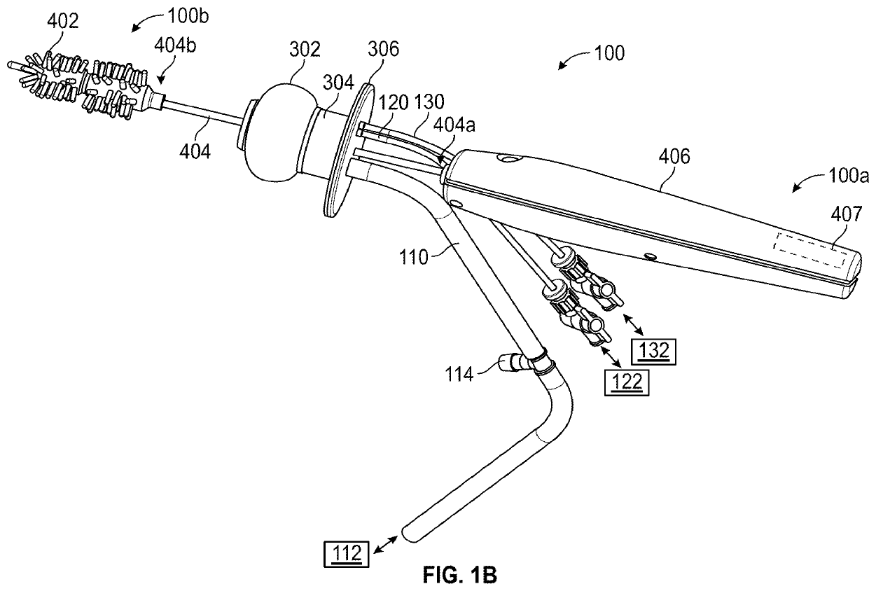

FIGS. 1A and 1B depict a treatment device 100 (also referred to herein as “device 100”) configured in accordance with several embodiments of the present technology. The device 100 can be configured for insertion through the anus into a lumen of an intestine (such as the colon) and disrupt, capture, and/or remove obstructive material, such as impacted fecal matter, from the intestinal lumen. As used herein, the act of “disrupting” fecal matter includes breaking the material into smaller pieces, modifying the shape of the material, stretching and/or elongating the material, compressing the material, permanently or temporarily repositioning all or a portion of the material, extracting all or a portion of the material, macerating the material, morcellating the material, softening the material, shaving the material, shearing the material, and/or any other action that modifies the shape, size, and/or position of the fecal matter.

The device 100 comprises a proximal region 100a configured to be positioned outside of the patient's body and manipulated by a user, and a distal region 100b configured to be positioned at a treatment site within a lumen of the intestine (e.g., the colon, the rectum, the anal canal, etc.) proximate fecal matter, such as impacted fecal matter. As used herein, the term “proximal” refers to a position that is closer to the clinician along a longitudinal axis of the device 100, and the term “distal” refers to a position that is farther from the clinician along the longitudinal axis of the device 100. As best shown in FIGS. 1A and 1B, the device 100 can comprise a hub 300 configured to be positioned across the patient's anus to help anchor and seal the device 100, and a disruptor 400 slidably received through the hub 300 and configured to engage fecal matter at the treatment site. The disruptor 400 comprises a proximal region including a handle 406, a distal region including a distal element 402 (only partially shown in FIG. 1A), and an elongate member 404 extending therebetween. The elongate member 404 can have a proximal portion 404a coupled to the handle 406 and a distal portion 404b (only visible in FIG. 1B) coupled to the distal element 402. The elongate member 404 can comprise a solid rod or may be a hollow shaft having a lumen extending therethrough. In some embodiments, including that shown in FIGS. 1A and 1B, the device 100 and/or disruptor 400 includes an energy source 407 (shown schematically) operably coupled to the distal element 402 (e.g., via the elongate member 404). Activation of the energy source energizes the distal element 402, thereby causing the distal element 402 to deliver energy to the fecal matter and dislodge a portion of the fecal matter.

In some embodiments, the energy source 407 is a motor, and activation of the motor causes the distal element 402 to move (e.g., vibrate, translate, and/or rotate), thereby agitating the fecal matter. The motor can be housed within the handle 406 and activated by an actuator 408 on the handle 406, such as a button, switch, knob, etc. In some embodiments, the motor is powered by a battery, which may be contained in the handle 406. Additionally, or alternatively, the motor may be configured to be electrically coupled to an external power source, such as a wall outlet. In these and other embodiments, the distal element 402 can comprise one or more ultrasound transducers and the energy source 407 comprises a pulse generator. The ultrasound transducer can convert the electrical signals created by the pulse generator into ultrasound signals that are configured to disrupt fecal matter at the treatment site. The ultrasound signals can vary in shape, duration, and length. For example, electrical signals from the pulse generator, and the corresponding ultrasound signals, may be a train of pulses of a fixed or variable frequency, duty-cycle, and duration. In some embodiments, the pulse generator and ultrasound transducer are configured to deliver acoustical waves having a frequency of about 10 kHz to 100 kHz, 10 kHz to 50 kHz, 10 kHz to 1000 kHz, at least 10 kHz, at least 20 kHz, at least 30 kHz, at least 40 kHz, or more than 1000 kHz. In some embodiments, the distal element 402 comprises one or more reflectors, and the system includes one or more transducers configured to be positioned outside the body and not in direct contact with the distal element 402.

Regardless of the form of energy (e.g., mechanical, ultrasonic, etc.), the intensity of the disruptive energy may be adjustable, for example via one or more actuators on the handle 406. In some embodiments, the device 100 does not include an energy source and the user moves the distal element 402 to dislodge the fecal matter manually.

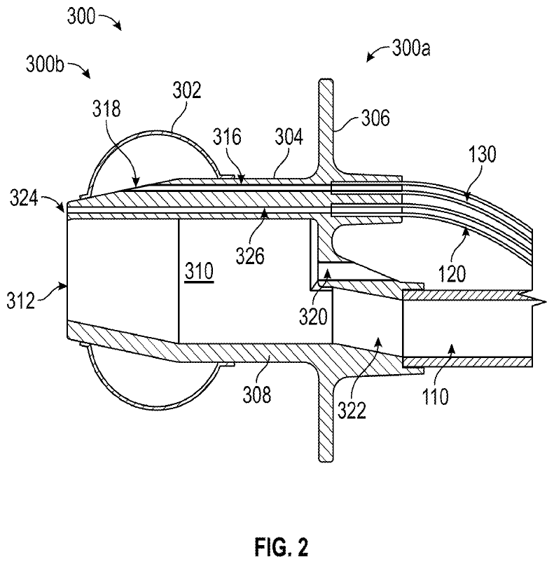

The device 100 can include one or more fluid channels extending between the proximal and distal regions 100a, 100b of the device 100 for infusing fluids to and/or removing fluids and fecal matter from the treatment site. For example, the device 100 can comprise a first channel 110 extending distally from a port at the proximal region 100a to an opening at the distal region of the device 100 (e.g., at or distal of the hub 300). The first channel 110 can be fluidly coupled to a negative pressure source 112 (e.g., a vacuum pump, a syringe, etc.) to apply a negative pressure at the treatment site to remove disrupted fecal matter present in the intestinal lumen (e.g., 0 to 800 mm Hg). In some embodiments, at least a portion of the first channel 110 extends through the body of the hub 300 (as shown in FIG. 2), and at least another portion of the first channel 110 extends through a tube coupled to and extending proximally away from a proximal portion of the hub 300. The tube can optionally include a flush port, such as a T-port 114. According to certain embodiments, the first channel 110 can be used both for aspiration and infusion of fluids. For example, the first channel 110 can be configured to be operably coupled to a fluid source.

In some embodiments, the device 100 comprises a second channel 120 extending distally from a port at the proximal region 100a of the device 100 to an opening at the distal region 100b of the device 100 (e.g., at or along the hub 300, at or along the elongate member 404, at or along the distal element 402, etc.). The second channel 120 can be configured to be fluidly coupled to a fluid source 122 to deliver a fluid through the second channel 120 to the treatment site. The fluid can be, for example, water, saline, mineral oil, water mixed with soap suds, a water-based solution with stool softener, and/or other non-toxic fluids. In some embodiments, at least a portion of the second channel 120 extends through the body of the hub 300 (as shown in FIG. 2), and at least another portion of the second channel 120 extends through a tube coupled to and extending proximally away from a proximal portion of the hub 300. According to several embodiments, the second channel 120 extends through the elongate member 404 and terminates at one or more openings at the distal portion 404b of the elongate member 404 and/or along the distal element 402. In some embodiments, the device 100 does not include a second channel 120.

The device 100 can further comprise a third channel 130 extending distally from a port at the proximal region 100a of the device 100 to an opening within an expandable portion of the hub 300. The third channel 130 can be configured to be fluidly coupled to a fluid source and/or a pressure source 132 to transform the expandable portion of the hub 300 from a collapsed state (shown in FIG. 1A) to an expanded state (shown in FIG. 1B). In some embodiments, at least a portion of the third channel 130 extends through the body of the hub 300 (as shown in FIG. 2), and at least another portion of the third channel 130 extends through a tube coupled to and extending proximally away from a proximal portion of the hub 300. According to certain embodiments, the device 100 does not include a third channel 130. It will be appreciated that the device 100 can include a single channel (which may be any one of the first, second, or third channels), two channels (which may be any two of the first, second, or third channels), three channels, or more than three channels.

FIG. 2 is a side cross-sectional view of the hub 300 with the disruptor 400 removed for ease of viewing the features of the hub 300. The hub 300 can include a hub body 308 having a proximal portion 300a configured to be positioned proximal of the anus at an extracorporeal location, a distal portion 300b configured to be positioned in the intestinal lumen, and an intermediate portion 304 between the proximal and distal portions 300a, 300b. The intermediate portion 304 can be configured to be positioned along the anal canal when the device 100 is in use. As such, the hub 300 can have a length that is longer than the length of the anal canal (e.g., at least 4 cm, at least 5 cm, at least 6 cm, at least 7 cm, or at least 8 cm). The hub body 308 can define an inner cavity 310 that is configured to receive all or a portion of the distal element 402 (FIGS. 1A and 1B). The inner cavity 310 may terminate distally at a distal opening 312 configured to receive at least a portion of the distal element 402 therethrough. In some embodiments, for example as shown in FIG. 2, a distal region of the hub body 308 can taper distally to facilitate introduction of the device through the anus. In other embodiments, the portion of the hub body 308 configured to be positioned within the patient's body can have a constant cross-sectional dimension.

In some embodiments, the distal portion 300b of the hub 300 includes an expandable portion 302 having a collapsed state and an expanded state. For example, the expandable portion 302 can include a membrane positioned around the circumference of the hub body 308 at the distal portion 300b of the hub 300. The hub body 308 can include an inflation lumen 316 configured to be fluidly coupled to a fluid source 132 (see FIGS. 1A and 1B) and that terminates distally at an opening 318 aligned with the membrane. As such, delivery of a fluid (i.e., a gas or a liquid) through the inflation lumen 316 causes the membrane to expand away from the hub body 308. Likewise, removal of a gas or liquid from the space between the membrane and the hub body 308 causes the membrane to collapse down, towards the hub body 308, thereby transforming the expandable portion 302 into the collapsed, low-profile state. In some embodiments, the inflation lumen 316 can be configured to be fluidly coupled with and/or comprise a portion of the third channel 130.

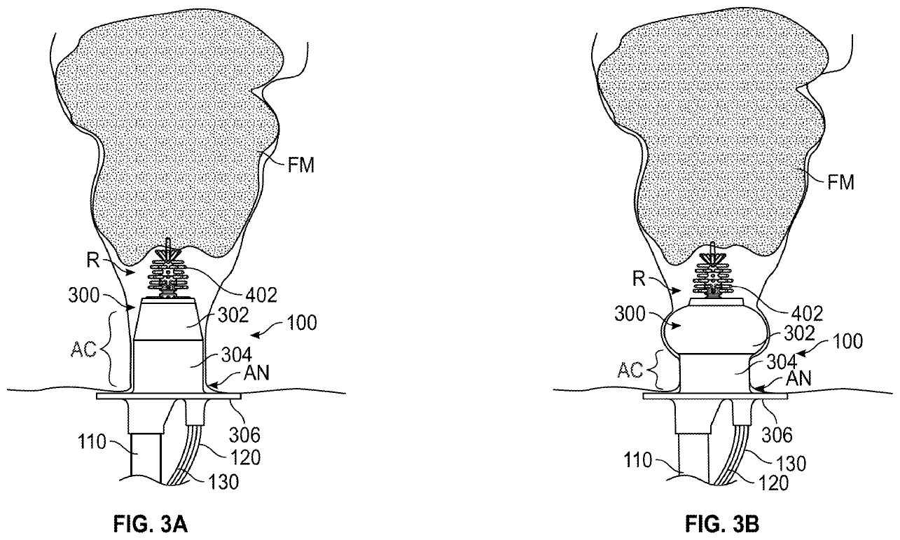

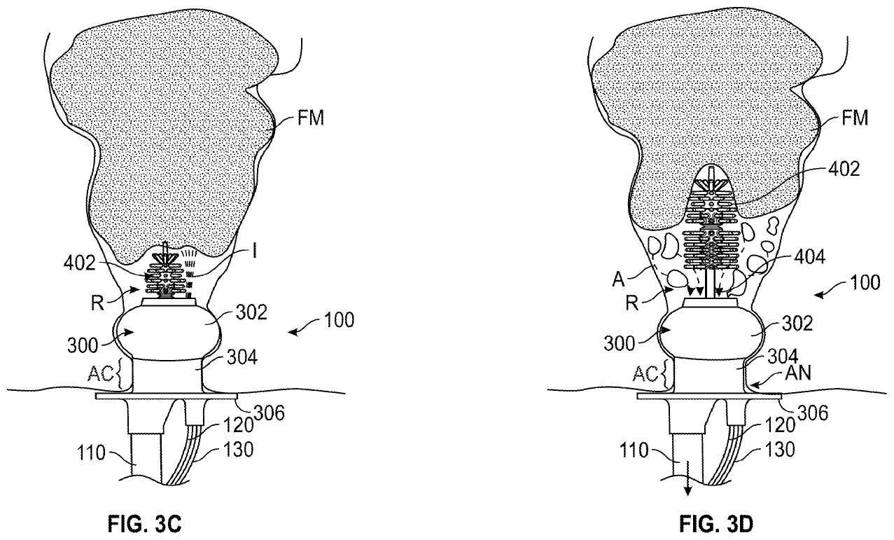

In the collapsed state (shown in FIG. 1A), the hub 300 has a smaller cross-sectional dimension and is configured for introduction through the anal canal into the intestinal (rectal) lumen. In the collapsed state, the hub 300 can have a cross-sectional dimension that is sized to allow comfortable insertion into the anus. For example, when the hub 300 is in the collapsed state, the hub 300 can have a cross-sectional dimension of at least 0.5 inches and no more than about 3 cm, or no more than about 5 cm. In the expanded state (shown in FIGS. 1B and 2), the expandable portion 302 is configured to conform and seal against the inner wall of the intestinal lumen (for example, as shown and described below with reference to FIGS. 3A-3D) to prevent or reduce leakage of intestinal fluids and/or fecal matter through the anus to an extracorporeal location. The compliant nature of the membrane and/or expandable portion 302 allows the hub 300 to seal against irregular geometry of the rectum just distal to the opening to the anal canal. Additionally, the hub 300 is configured to cover the distal element 402 (e.g., by receiving all or a portion of the distal element 402 within the inner cavity 310) during insertion and removal of the device 100, thus acting as a barrier and protecting the mucosa of the gastrointestinal tract.

Referring still to FIG. 2, the hub 300 can include a broad proximal element 306 configured to be positioned proximal of the anus at an extracorporeal location while the device 100 is in use. The proximal element 306 can have a cross-sectional dimension that is greater than a diameter of the anus such that the proximal element 306 is prevented from moving distally through the anus. As a result, the proximal element 306 limits a depth at which the hub 300 can be inserted into the rectum. In FIG. 2, the proximal element 306 has a substantially fixed cross-sectional area. In other embodiments, the proximal element 306 can be expandable and/or inflatable.

The hub body 308 can include one or more lumens extending therethrough. For example, as shown in FIG. 2, the hub 300 can include a disruptor lumen 320 that is configured to slidably receive the elongate member 404 of the disruptor therethrough. Additionally, or alternatively, the hub body 308 can include an aspiration lumen 322 configured to be fluidly coupled to and/or comprising a portion of the first channel 110. In some embodiments, the aspiration lumen 322 terminates distally at the inner cavity 310 of the hub body 308. In other embodiments, the aspiration lumen 322 can terminate at an outer surface of the hub body 308. Additionally, or alternatively, the hub body 308 can include an infusion lumen 326 configured to be fluidly coupled to and/or comprising a portion of the second channel 120. In some embodiments, the infusion lumen 326 terminates distally at the distal face of the hub body 308. As a result, fluid delivered through the infusion lumen 326 is ejected into a space distal of the hub 300 and near the distal element 402. As previously mentioned, in some embodiments the hub 300 may comprise an inflation lumen 316 for delivering a fluid to a space between the membrane and the outer surface of the hub body 308. According to several embodiments of the technology, the hub 300 may include fewer than four lumens (e.g., three lumens, two lumens, one lumen, etc.). For example, the hub 300 may include only a disruptor lumen 320 and a single other lumen. The single other lumen can be used for both aspiration and fluid infusion.

FIGS. 3A-3D depict a method of using the device 100 to dislodge and/or remove impacted fecal matter from a patient's rectum. To begin a procedure, a patient is positioned on their side as shown in FIG. 3A. This position advantageously enables easy insertion of the device 100.

As shown in FIG. 3A, the user first inserts the hub 300 with the expandable portion 302 in the collapsed state through the anus AN into the rectum R until the proximal element 306 is seated against the tissue surrounding an exterior aspect of the anus AN. During insertion, the distal element 402 of the disruptor 400 can be positioned at least partially within an interior cavity of the hub 300 with a distal portion of the distal element 402 extending distally beyond a distal end of the hub 300. The distal portion of the distal element 402 can be tapered distally, for example, to facilitate insertion through the anus AN and anal canal AC. Likewise, the distal portion of the hub 300 can be tapered distally, at least when the expandable portion 302 is in a collapsed state. In some embodiments, the hub 300 and/or distal element 402 are lubricated prior to insertion. According to several embodiments, the entire distal element 402 is housed within the hub 300 during insertion.

As shown in FIG. 3A, once the hub 300 is inserted with the proximal element 306 abutting the tissue surrounding the anus AN, all or a portion of the intermediate portion 304 of the hub 300 is positioned within the anal canal AC and all or substantially all of the collapsed expandable portion 302 and protruding distal element 402 are positioned within the rectum R. In some embodiments, the hub 300 can be configured such that a length of the intermediate portion 304 approximates a length of the anal canal AC, thereby positioning a proximal end of the expandable portion 302 in the rectum R, just distal of the distal end of the anal canal AC (e.g., at the anorectal junction). As discussed in greater detail herein, the hub 300 can have a maximum outer diameter configured to enable comfortable insertion (e.g., up to approximately 3 cm, or up to approximately 4 cm, or up to approximately 5 cm), and the length of the hub 300 can be configured to be longer than the length of the anal canal AC (e.g., greater than approximately 4 cm, great than approximately 5 cm, or greater than approximately 6 cm).

Next, as shown in FIG. 3B, the user holds the hub 300 in place against the anus AN while expanding the expandable portion 302 against the portion of the rectum R just distal of the anorectal junction to secure the hub 300 such that the hub 300 remains in position during the remainder of the procedure without the user having to hold it in position. Thus, when the expandable portion 302 is in an expanded state, the anal canal tissue is positioned between the expandable portion 302 and the proximal element 306. The expandable portion 302 and proximal element 306 work together to secure the hub 300 throughout the procedure, such as during longitudinal and rotational motion of the distal element 402, injection of irrigation fluid, and aspiration of irrigation fluids and fecal matter, etc. The expandable portion 302 further prevents fecal matter FM from leaking out of the rectum R to maintain a clean procedure. For example, the expandable portion 302 can have a diameter such that it is configured to expand into apposition with the inner surface of the rectal wall and substantially seal against the rectal wall to prevent egress of fluids proximally into the anal canal AC. As shown in FIG. 3B, in many embodiments, the rectal wall conforms to the shape of the expandable portion 302. In some embodiments, the expandable portion 302 can be slightly oversized relative to a diameter of the rectum just distal of the anal canal such that the expandable portion 302 exerts a radially outward force on the rectum during the procedure.

As shown in FIG. 3C, at this stage the user can inject a fluid through the device 100 into the rectum R. In some embodiments, the fluid can be configured to soften the impacted fecal matter FM to facilitate removal of the fecal matter. Examples of such fecal modifying fluids include water, saline, mineral oil, water mixed with soap, a water-based solution with stool softener, and other fluids. The fluid can be delivered through the second channel and second port. Additionally, or alternatively, the fluid can be delivered through the first channel and opening. Either way, the fluid passes from an extracorporeal location (such as a syringe or other fluid source) through the hub 300 and into the rectum R. Infusion of one or more fluids can occur before, during, and/or after actuation of the distal element 402 (which is be translation, rotation, or vibration), or in some cases not at all. Likewise, infusion of one or more fluids can occur before, during, and/or after aspiration, and aspiration can occur before, during and/or after actuation of the distal element 402 (which is be translation, rotation, or vibration), or in some cases not at all.

Referring now to FIG. 3D, the distal element 402 can be advanced distally to engage or further engage the fecal matter FM. As shown, the user can rotate and/or translate (automatically or manually) the distal element 402 to disrupt or otherwise break up the fecal matter into smaller and/or softer pieces. Aspiration A and/or infusion of fluids can occur while the distal element 402 is activated and breaking up fecal matter to remove the treated fecal matter from the treatment site.

The mechanical or ultrasonic energy delivered by the distal element, the introduction of irrigation liquids, and the application of vacuum through the vacuum port, work in concert to disimpact the impacted stool and/or fecal matter, creating small particles of fecal matter together with a slurry fluid. The elongate member 404 can be slowly advanced distally, in combination with rotation, to continue breaking the impacted stool into small pieces and eventually, slurry. The vacuum port also has a setting where the port can be cleared and flushed in the situation of large pieces of fecal matter are obstructing this connection.

At the conclusion of the procedure, when all the stool particles and liquid stool slurry have been removed, the user can pull the elongate member 404 proximally to withdraw the distal element 402 back into the interior cavity of the hub 300. The user can then collapse and/or deflate the expandable portion so that the hub 300 can be pulled proximally through the anus.

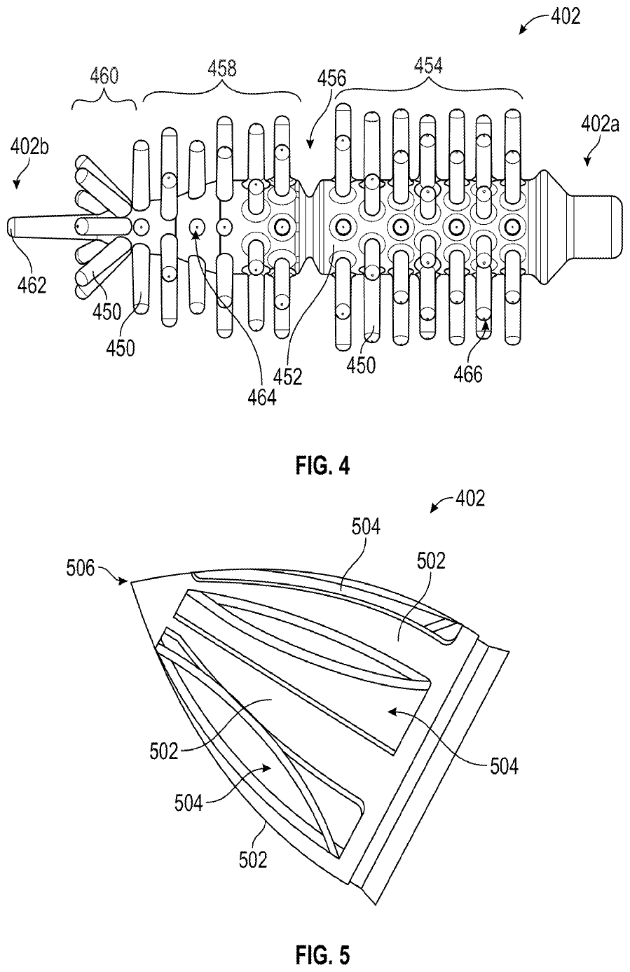

FIGS. 4, 5, 6, 7, 8, 9, 10, 11, 12, 13, 14, 15 and 16 show various distal elements configured for use with the disruptors of the treatment devices of the present technology. For any of the following embodiments, the distal element is configured to penetrate any inspissated fecal matter without causing any trauma to the surrounding tissue of the anus and rectum.

As shown in FIG. 4, in some embodiments the distal element 402 comprises a shaft 452 and a plurality of projections 450 extending away from the shaft 452. The distal element 402 can include a first zone 454, a second zone 458, and a third zone 460, each comprising a plurality of projections 450. The distal element 402 can comprise a bending zone 456 between the first and second zones 454, 458 and along which a diameter of the shaft 452 is narrowed. It can be advantageous to include such a bending zone as it provides the distal element 402 with a greater flexibility and improves the distal element's ability to adapt to the curvature of the rectum, colon, or other section of the intestines. Moreover, the projections 450 and/or the entirety of the distal element 402 can be made of a soft, durable, biocompatible material, such as nylon, polyester, or polyethylene, and/or other polymers.

Referring still to FIG. 4, at least a portion of the shaft 452 along the second zone 458 can taper distally until terminating at the third zone 460, along which the projections 450 extend at a non-90-degree angle relative to the longitudinal axis of the distal element 402 (unlike the projections 450 along the first and second zones 454, 458 which extend away from the shaft 452 at substantially 90 degrees relative to the longitudinal axis). In some embodiments, the distal element 402 includes a single distal projection 462.

In some embodiments the shaft 452 has a lumen extending therethrough, and the lumen is configured to be fluidly coupled to a fluid source and/or negative pressure source. The distal element 402 can include one or more openings 466 on or along one, some, or all of the projections 450. Additionally or alternatively, the distal element 402 can include one or more openings 464 along the shaft 452. Fluid delivered through the lumen of the shaft can be injected at the treatment site through the openings 464, 466, which can be in addition to, or instead of, an infusion channel in the hub 300. In some embodiments, the distal element 402 is solid (e.g., not hollow) and/or does not include any openings.

FIG. 5 depicts another distal element 402 for use with the devices 100 of the present technology. The distal element 402 can be coupled to the distal end of the elongate member 404 such that movement of the elongate member 404 (e.g., translation, rotation, and/or mechanical or ultrasonic vibration) causes a corresponding movement in the distal element 402. The distal element 402 can be tapered along all or a portion of its length, and can include a plurality of struts 502 separated by a plurality of windows 504. In some embodiments, the windows 504 are in fluid communication with the elongate member 404, and the lumen of the elongate member 404 can be configured to be coupled to a fluid source and/or a negative pressure source. As such, fluid can be delivered through the windows 504 of the distal element 402 to the treatment site, and fluids and/or fecal matter at the treatment site can be removed proximally through the distal element 402 and the elongate member 404. As shown in FIG. 5, in some embodiments the distal element 402 can include a sharpened tip 506. In other embodiments, the tip is curved or otherwise atraumatic (for example, like tip 706 in FIG. 7). One or both edges of the struts 502 can be sharpened, or may be dull. Either way, rotation of the distal element 402/struts 502 at the treatment site can shear the fecal matter to make it easier to remove the fecal matter through the windows 504. In some embodiments, the elongate member 404 is a solid rod, and infusion and/or aspiration occurs exclusively through the hub 300.

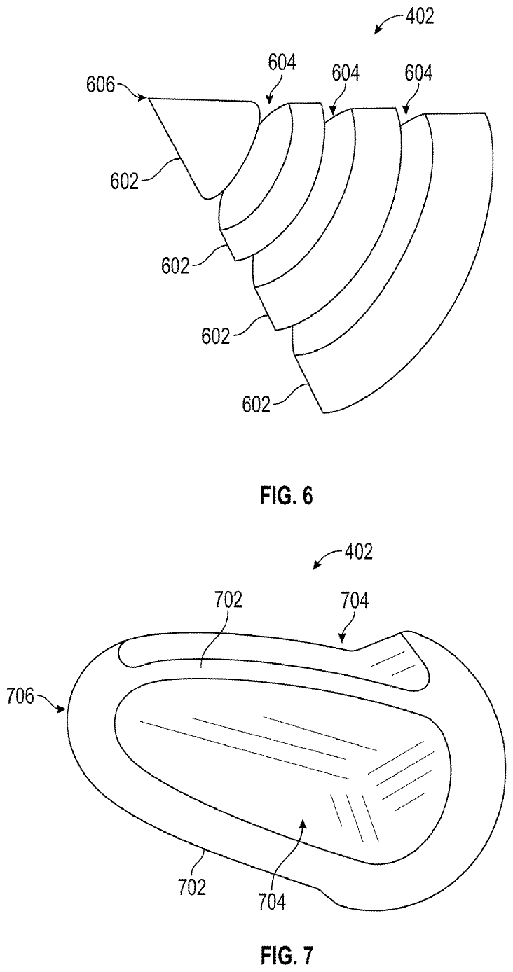

In FIG. 6, the distal element 402 is again tapered and terminates at a sharpened tip 606. In other embodiments, the distal element 402 need not be tapered along its entire length and/or includes a rounded or otherwise atraumatic tip. The distal element 402 can be coupled to the distal end of the elongate member 404, such that movement of the elongate member 404 (e.g., translation, rotation, and/or vibration) causes a corresponding movement in the distal element 402. The distal element 402 also comprises a circumferentially winding groove 604 that creates a plurality of discs 602 that decrease in diameter in a distal direction. The edges of the discs 602 can be sharpened or dull.

FIG. 7 depicts another distal element 402 configured in accordance with the present technology. The distal element 402 of FIG. 7 is again tapered and terminates at a rounded tip 706. In other embodiments, the distal element 402 need not be tapered along its entire length and/or includes a sharpened tip. The distal element 402 can be coupled to the distal end of the elongate member 404 such that movement of the elongate member 404 (e.g., translation, rotation, and/or vibration) causes a corresponding movement in the distal element 402. The distal element 402 of FIG. 7 can include longitudinal ridges 702 that are separated by recesses 704 about a circumference of the distal element 402. The inner surfaces of the recesses 704 can be substantially flat and/or linear. Unlike the windows 504 of the distal element 402 of FIG. 5, the recesses 704 of the distal element 402 in FIG. 7 do not open into an interior region.

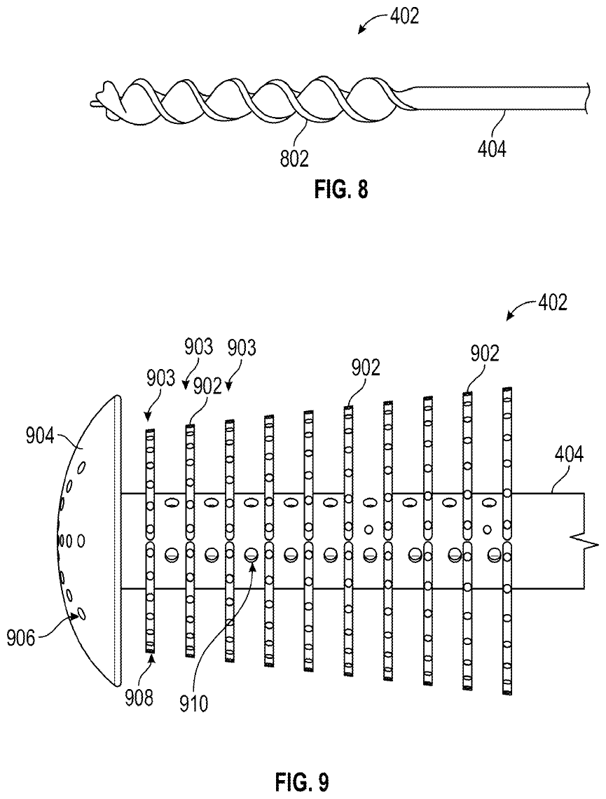

FIG. 8 depicts another embodiment of a distal element 402 comprising a rod or tube with a spiral-shaped distal portion 802 configured to penetrate the fecal matter. In this embodiment, the distal element may come to a point 806 or it can have tips that comprise the distal elements 402 in FIGS. 5, 6 and 7. In this embodiment, the distal element 402 is configured to create a hole in the fecal matter as the distal element 402 rotates. In some embodiments, the distal element 402 can have one or more holes along the distal portion 802.

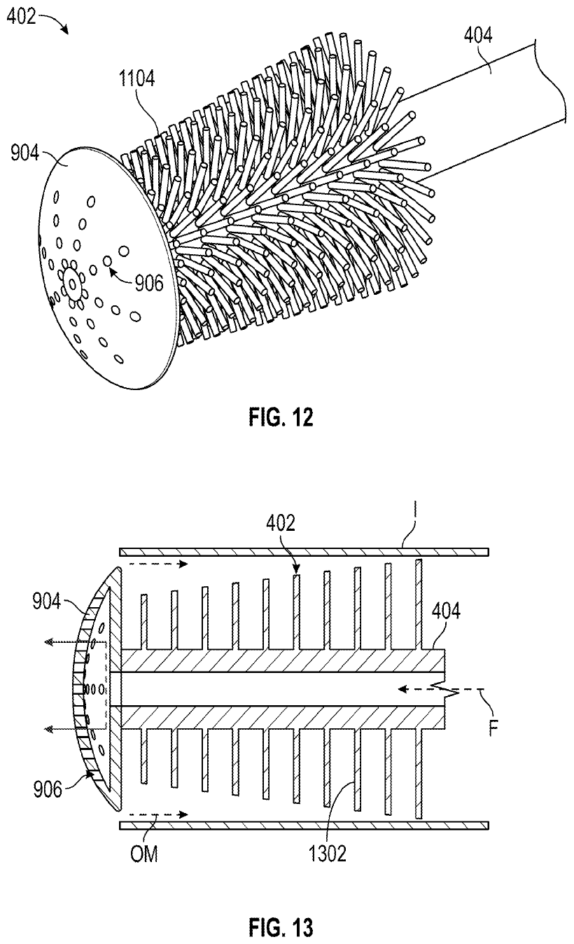

FIG. 9 shows another embodiment of a distal element 402 comprising a plurality of projections 902 extending away from a distal portion of the elongate member 404. FIG. 12 is a perspective view of FIG. 9. The individual projections 902 can be arranged spaced apart about a circumference of the elongate member 404, and spaced apart along the longitudinal axis of the elongate member 404. In some embodiments, the projections 902 are arranged in groups 903 that are spaced apart along the longitudinal axis of the elongate member 404 (only three of the groups 903 are labeled in FIG. 9 for ease of viewing). The individual projections 902 within each group 903 are located at substantially the same axial location along the elongate member 404 but extend away from the elongate member 404 at different circumferential positions and at different angles. In some embodiments, the projections 902 are not arranged in longitudinally spaced groups and instead are randomly disposed along the longitudinal axis.

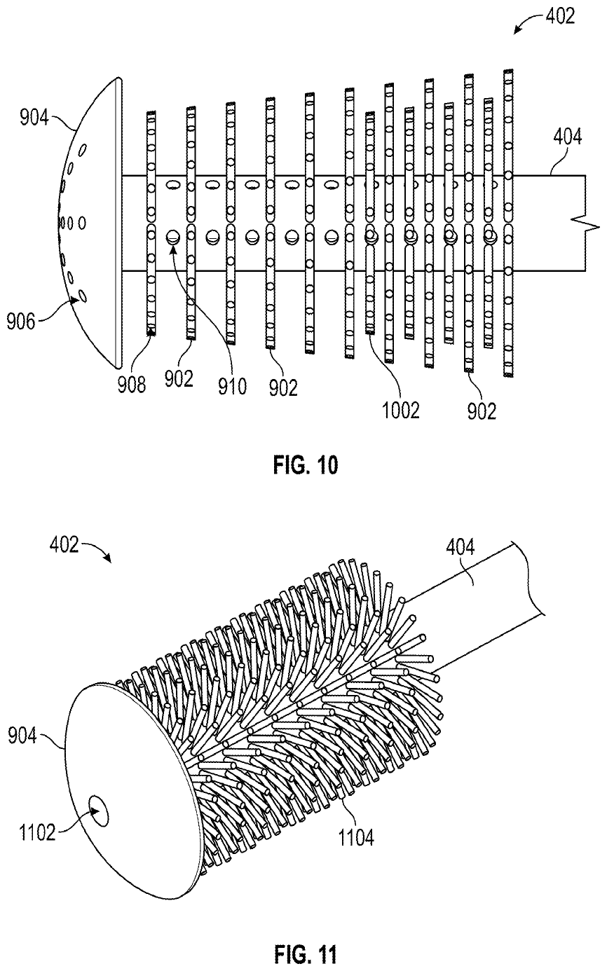

The distal element 402 further comprises an atraumatic tip 904 that presents a broad, curved, distally facing surface. The projections 902 can be made of soft, durable, biocompatible materials, such as nylon, polyester, or polyethylene, and/or other polymers. The projections 902 can be the same length or may vary in length. For example, as shown in FIG. 9, the lengths of the projections 902 can decrease in a distal direction. In some embodiments, the projections 902 have different lengths but do not necessarily increase or decrease in a particular direction. In some embodiments, for example as shown in FIG. 10, certain portions of the distal element 402 can have a higher density of projections 902. The projections 902 may be denser along a distal portion, a more central portion, or a proximal portion (as shown in FIG. 10). In some embodiments, the projections 902 closer to the distal end of the distal element can be shorter and/or stouter and slightly further apart (as compared to the more proximal projections). This way, the more distal projections 902 can be configured to break up the fecal matter into larger chunks, which are then broken down into smaller pieces by the longer and/or more flexible and more closely spaced proximal projections 902. In these and any embodiments herein, a stiffness of the individual projections 902 can vary along a length of the distal element 402. The projections 902 can become progressively more flexible in a proximal direction, or may get stiffer in a proximal direction. Likewise, the groups 903 of projections 902 can get progressively more flexible in a proximal direction, or may get stiffer in a proximal direction.

In some embodiments the elongate member 404 has a lumen extending therethrough, and the lumen is configured to be fluidly coupled to a fluid source and/or negative pressure source. The distal element 402 can include one or more openings 908 on or along one, some, or all of the projections 902. Additionally, or alternatively, the distal element 402 can include one or more openings 910 along the portion of the elongate member 404 coincident with the projections 902. Additionally, or alternatively, the distal element 402 can include one or more openings 906 at the tip 904. Fluid delivered through the lumen of the elongate member 404 can be injected at the treatment site through the openings 906, 908, 910, which can be in addition to or instead of an infusion channel in the hub 300. In some embodiments, the distal element 402 is solid (e.g., not hollow) and/or does not include any openings.

FIG. 11 shows an embodiment of a distal element 402 that is substantially the same as the distal element 402 shown in FIG. 9, except in FIG. 11 the length of the projections 1104 are the same along the length of the distal element 402, and the distal tip 904 has a single opening 1102. In other embodiments, the distal tip 904 can have a plurality of openings.

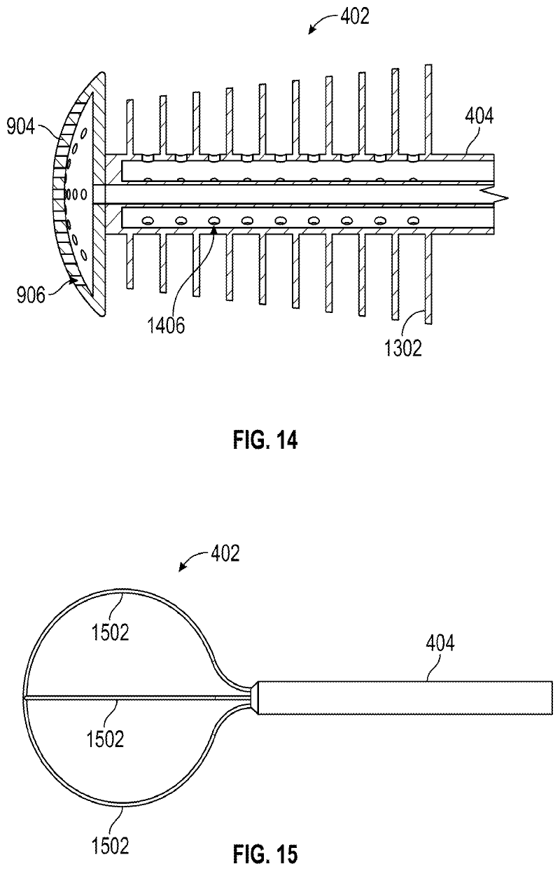

FIG. 13 depicts the distal element 402 of FIG. 12 while infusing fluid F and aspirating obstructive material OM. FIG. 14 shows another variation in which the elongate member 404 has openings 1406 in addition to the openings 906 of the distal tip 904.

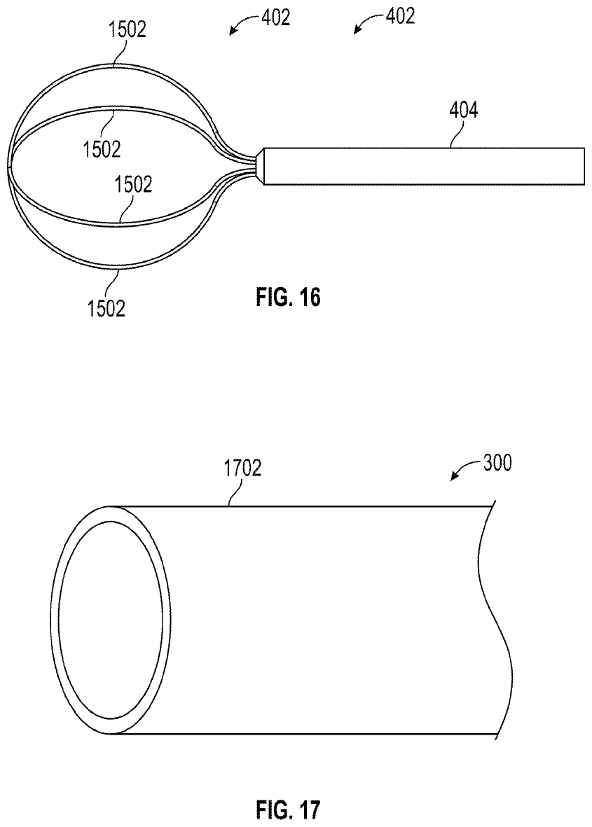

Referring to FIG. 15, in some embodiments the distal element 402 can comprise a basket comprising a plurality of struts 1502 configured to penetrate fecal matter. The basket can be made of one or more wires, or may be a laser-cut stent. The struts 1502 are shaped to present smooth and/or curved surfaces to the mucosa of the wall of the rectum. The wires can be configured to penetrate the fecal matter. FIG. 16 shows a different view of the distal element 402 of FIG. 15 after the elongate member has been rotated.

All of the distal elements 402 disclosed herein are configured to be received partially or completely within the inner cavity of a hub of the present technology (for example, inner cavity 310 of the hub 300 in FIG. 2). Various other shapes and configurations may be used to facilitate penetration, dislodgement, or breakup of the fecal matter (e.g., fecal inspissated mass).

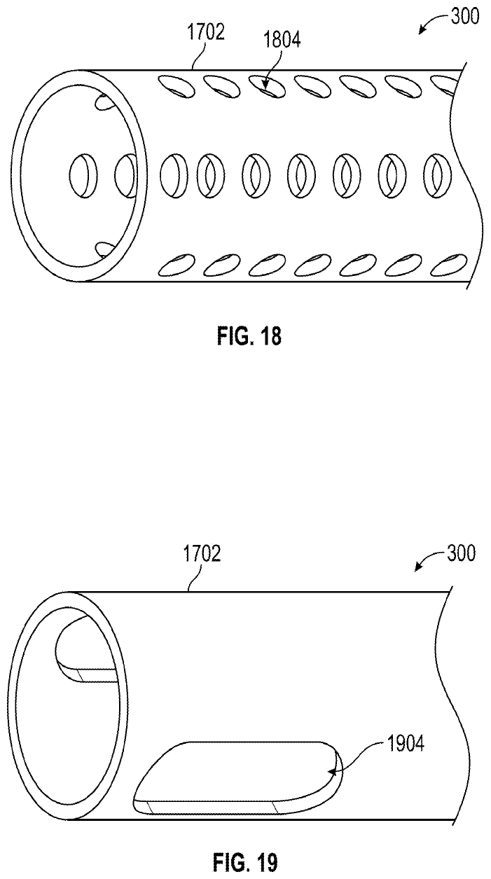

FIGS. 17, 18 and 19 show various hubs configured for use with the treatment devices of the present technology. These hubs can be extensions of any of the hubs disclosed herein, or separate elongate elements that are introduced through the hubs of the present technology. In any case, the hubs of FIGS. 17, 18 and 19 can be positioned distal of the expandable portion of a respective treatment device to better allow suction of fecal matter into the hub. FIG. 17 shows a tubular hub 1802 where the distal element 402 is introduced through a lumen in the hub. The lumen can be configured to be fluidly coupled to a negative pressure source and/or a fluid source for aspiration and/or infusion of fluids, respectively. FIG. 18 is an embodiment of the hub with a series of holes 1804 spaced apart along its sidewall. In use, the holes 1804 are generally perpendicular to the axis of the rectum and provide more points of entry of stool slurry into the hub. The holes can be of various dimensions, both small and large. The holes along with the suction from the aspiration channel allows dispersed fecal matter to move through the holes. Referring to FIG. 19, the holes 1904 in the hub can also be of various shapes or openings, further enhancing the process of suction of the dispersed fecal material into the hub, rotation of the elongate member, and the associated movement of the distal element in breaking up the fecal matter.

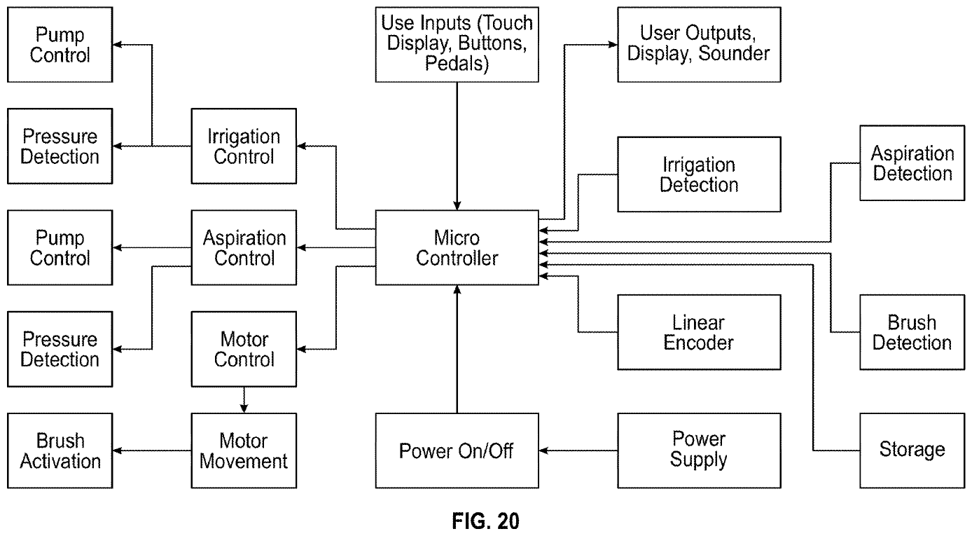

FIG. 20 is an overview diagram demonstrating the organization of the control system. The control system includes a display or user interface, a microcontroller, a motherboard, irrigation control, aspiration control, motor control, user outputs, system temperature, brush detection, and battery-type power source and various inputs and outputs. In some embodiments, the device includes one or more sensors that are operatively coupled to the control system. The sensors can, for example, provide torque feedback to the control system that, in response, may shut down the motor in order to prevent injury. In some embodiments, the device can include one or more sensors configured to detect fluid levels so that, in response, the control system may release additional irrigation liquids into the hub/rectum to maintain an appropriate amount of liquid inside the rectum. In certain embodiments, the device can include or more sensors configured to detect the level of maceration of the stool and, in response, the control system can turn the motor on/off and/or turn the suction on/off. These and other feedback loops can be utilized alone or together to improve the user experience, minimize potential injury, and increase fecal matter extraction.

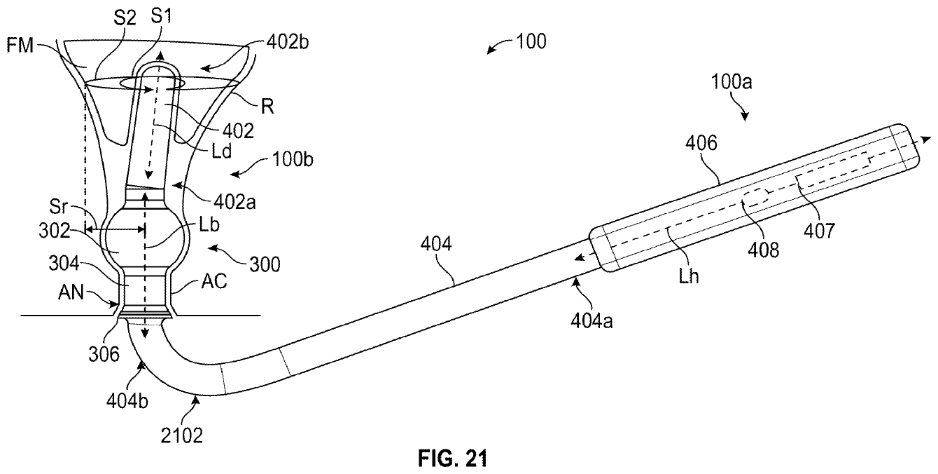

FIG. 21 depicts a treatment device 100 configured in accordance with several embodiments of the present technology, shown positioned in a patient's rectum R. The device 100 can be configured for insertion through the anus AN into a lumen of an intestine (such as the colon) and disrupt, capture, and/or remove obstructive material, such as impacted fecal matter FM, from the intestinal lumen. The device 100 comprises a proximal region 100a configured to be positioned outside of the patient's body and manipulated by a user, and a distal region 100b configured to be positioned at a treatment site within a lumen of the intestine (e.g., the colon, the rectum R, the anal canal AC, etc.) proximate fecal matter FM, such as impacted fecal matter. The device 100 can further include a handle 406 at the proximal region 100a, an elongate member 404 extending between the proximal and distal regions 100a, 100b of the device 100, a hub 300 at a distal end portion 404b of the elongate member 404, and a distal element 402 extending distally from a distal end portion of the hub 300.

As previously described, the hub 300 can be configured for positioning across the patient's anus AN to help anchor and seal the device 100. The hub 300 can include a hub body having a proximal portion configured to be positioned proximal of the anus AN at an extracorporeal location, a distal portion configured to be positioned in the intestinal lumen, and an intermediate portion 304 between the proximal and distal portions. The intermediate portion 304 can be configured to be positioned along the anal canal AC when the device 100 is in use. In some embodiments, the distal portion of the hub 300 includes an expandable portion 302 having a collapsed state and an expanded state. It will be appreciated that the description of the hub 300 provided above with reference to FIGS. 1-19 similarly applies to the hub 300 of FIG. 21.

The elongate member 404 can have a proximal portion 404a coupled to the handle 406 and a distal portion 404b coupled to the hub 300. The elongate member 404 can comprise a solid rod or may be a hollow shaft having a lumen extending therethrough. As shown in FIG. 21, the elongate member 404 may include a bend 2102 along its length. The bend 2102 can position a longitudinal axis Ld of the distal element 402 at an angle relative to the longitudinal axis Lh of the handle 406 and/or the longitudinal axis Lb of the hub 300. The angle provided by the bend 2102 can make it easier for the user to contact the rectal wall with sufficient force with the distal element 402 during manual rotation/swirling of the distal element 402 (via rotation of the handle 406 by the user).

In some embodiments, including that shown in FIG. 21, the device 100 and/or handle 406 optionally includes an energy source 407 (shown schematically) operably coupled to the distal element 402 (e.g., via wires extending through the elongate member 404). Activation of the energy source 407 energizes the distal element 402, thereby causing the distal element 402 to deliver energy to the fecal matter FM and dislodge a portion of the fecal matter FM. For example, the energy source 407 can be coupled to a motor, and activation of the motor causes the distal element 402 to move (e.g., vibrate, translate, rotate, swirl, etc.), thereby agitating the fecal matter FM. The motor can be housed within the handle 406 and activated by an actuator 408 on the handle 406, such as a button, switch, knob, etc. In some embodiments, the motor is powered by a battery, which may be contained in the handle 406. Additionally, or alternatively, the motor may be configured to be electrically coupled to an external power source, such as a wall outlet. In these and other embodiments, the distal element 402 can comprise one or more ultrasound transducers and the energy source 407 comprises a pulse generator. The ultrasound transducer can convert the electrical signals created by the pulse generator into ultrasound signals that are configured to disrupt fecal matter at the treatment site. The ultrasound signals can vary in shape, duration, and length. For example, electrical signals from the pulse generator, and the corresponding ultrasound signals, may be a train of pulses of a fixed or variable frequency, duty-cycle, and duration. In some embodiments, the pulse generator and ultrasound transducer are configured to deliver acoustical waves having a frequency of about 10 kHz to 100 kHz, 10 kHz to 50 kHz, 10 kHz to 1000 kHz, at least 10 kHz, at least 20 kHz, at least 30 kHz, at least 40 kHz, or more than 1000 kHz. In some embodiments, the distal element 402 comprises one or more reflectors, and the system includes one or more transfucers configured to be positioned outside the body and not in direct contact with the distal element 402.

Regardless of the form of energy (e.g., mechanical, ultrasonic, etc.), the intensity of the disruptive energy may be adjustable, for example via one or more actuators on the handle 406. In some embodiments, the device 100 does not include an energy source and the user moves the distal element 402 (by manipulating the handle 406 and/or elongate member 404) to dislodge the fecal matter manually.

As shown in FIG. 21, the distal element 402 can extend distally from a distal portion of the hub 300 and have an elongated shape with a curved, distally facing surface at its distal end. In some embodiments, the distal element 402 can have a cross-sectional dimension that tapers distally, for example to facilitate insertion through the anus AN and anal canal AC. According to several examples, the distal element 402 has a substantially constant diameter along its length. The distal element 402 can be substantially straight (as shown), or may be curved along all or a portion of its length.

The distal element 402 has a proximal end portion 402a, a distal end portion 402b, and a longitudinal axis Ld extending therebetween. While FIG. 21 shows a configuration of the distal element 402 in which the longitudinal axis Ld is angled relative to the longitudinal axis Lb of the hub 300 (e.g., statically or during a swirling motion, as described below), at any point during the procedure the longitudinal axis Ld may be aligned with the longitudinal axis Lb of the hub 300 (for example, when the distal element 402 is inserted through the anus AN into the rectum R).

In some embodiments, the distal element 402 can be configured to move in a circular motion and/or swirl while positioned in the rectum R (see arrows S1 and S2 in FIG. 21) to help disrupt the fecal matter FM and/or to induce rectal muscle constriction. For example, in those embodiments including an energy source 407 and/or motor, activation of the energy source 407 can cause the distal element 402 to swirl. The distal element 402 can be angled relative to the hub 300 during the swirling motion, with the distal end portion 402b of the distal element 402 radially farther from the longitudinal axis Lh of the hub 300 than the proximal end portion 402a. The swirl radius Sr, measured as the radial distance between the longitudinal axis Lb of the hub 300 and the distal end portion 402b of the distal element 402, can be varied. For example, in some cases, the distal element 402 swirls S1 at a swirl radius Sr that causes no or minimal forces to be exerted on the rectal wall by the distal end portion 402b. Additionally or alternatively, the distal element 402 can swirl S2 at a swirl radius Sr large enough such that the distal element 402 exerts a force on the rectal wall sufficient to induce rectal muscle constriction. As shown in FIG. 21, the rectum can narrow proximally, such that the distance between the proximal end portion 402a of the distal element 402 and the rectal wall is less than the distance between the distal end portion 402b and the rectal wall. As such, depending on the degree of narrowing, which can vary from patient to patient, swirling of the distal element 402 can cause all or a portion of the distal element 402 to contact the rectal wall. In some cases, the distal element 402 can be configured to vibrate while swirling (for example, by activation of the energy source 407).

In certain cases, the swirling motion can be achieved by moving the handle 406 in a circular motion, which may occur while vibratory and/or a swirling motion is provided by the energy source, or without additional energy inputs. During manual swirling, the longitudinal axis Ld of the distal element 402 may be aligned with the longitudinal axis Lb of the hub 300, or may be angled relative to the longitudinal axis Lb of the hub 300.

The device 100 can include one or more fluid channels extending between the proximal and distal regions 100a, 100b of the device 100 for infusing fluids to and/or removing fluids and fecal matter from the treatment site. For example, the device 100 can comprise a first channel extending distally from a port at the proximal region 100a to one or more openings at the distal region of the device 100 (e.g., at the hub 300, at the distal tip of the distal element 402 and/or along the sidewalls of the distal element 402, etc.). In some embodiments, the first channel can be fluidly coupled to a negative pressure source (e.g., a vacuum pump, a syringe, etc.) to apply a negative pressure at the treatment site to remove disrupted fecal matter present in the intestinal lumen (e.g., 0 to 800 mm Hg). At least a portion of the first channel may extend through the body of the hub 300, and at least another portion of the first channel extends through a tube coupled to and extending proximally away from a proximal portion of the hub 300. The tube can optionally include a flush port, such as a T-port. According to certain embodiments, the first channel can be used both for aspiration and infusion of fluids. For example, the first channel can be configured to be operably coupled to a fluid source.

In some embodiments, the device 100 comprises a second channel extending distally from a port at the proximal region 100a of the device 100 to an opening at the distal region 100b of the device 100 (e.g., at or along the hub 300, at the distal tip of the distal element 402 and/or along the sidewalls of the distal element 402, etc.). The second channel can be configured to be fluidly coupled to a fluid source to deliver a fluid through the second channel to the treatment site. The fluid can be, for example, water, saline, mineral oil, water mixed with soap suds, a water-based solution with stool softener, and/or other non-toxic fluids. In some embodiments, at least a portion of the second channel extends through the body of the hub 300, and at least another portion of the second channel extends through a tube coupled to and extending proximally away from a proximal portion of the hub 300. According to several embodiments, the second channel extends through the elongate member 404 and terminates at one or more openings along the distal element 402. In some embodiments, the device 100 does not include a second channel.

The device 100 can further comprise a third channel extending distally from a port at the proximal region 100a of the device 100 to an opening within an expandable portion of the hub 300. The third channel can be configured to be fluidly coupled to a fluid source and/or a pressure source to transform the expandable portion 302 of the hub 300 from a collapsed state (not shown in FIG. 21) to an expanded state. In some embodiments, at least a portion of the third channel extends through the body of the hub 300, and at least another portion of the third channel extends through a tube coupled to and extending proximally away from a proximal portion of the hub 300. According to certain embodiments, the device 100 does not include a third channel. It will be appreciated that the device 100 can include a single channel (which may be any one of the first, second, or third channels), two channels (which may be any two of the first, second, or third channels), three channels, or more than three channels.

To dislodge and/or remove impacted fecal matter from a patient's rectum, the patient is positioned on their side (for example, as shown in FIG. 3A). This position enables easy insertion of the device 100. The user then inserts the distal element 402 and hub 300 (with the expandable portion 302 in the collapsed state) through the anus AN into the rectum R until the proximal element 306 is seated against the tissue surrounding an exterior aspect of the anus AN.

Once the hub 300 is inserted with the proximal element 306 abutting the tissue surrounding the anus AN, all or a portion of the intermediate portion 304 of the hub 300 is positioned within the anal canal AC and all or substantially all of the collapsed expandable portion 302 and protruding distal element 402 are positioned within the rectum R. In some embodiments, the hub 300 can be configured such that a length of the intermediate portion 304 approximates a length of the anal canal AC, thereby positioning a proximal end of the expandable portion 302 in the rectum R, just distal of the distal end of the anal canal AC (e.g., at the anorectal junction). As discussed in greater detail herein, the hub 300 can have a maximum outer diameter configured to enable comfortable insertion (e.g., up to approximately 3 cm, or up to approximately 4 cm, or up to approximately 5 cm), and the length of the hub 300 can be configured to be longer than the length of the anal canal AC (e.g., greater than approximately 4 cm, great than approximately 5 cm, or greater than approximately 6 cm).

The method continues with the user holding the hub 300 in place against the anus AN while expanding the expandable portion 302 against the portion of the rectum R just distal of the anorectal junction to secure the hub 300 such that the hub 300 remains in position during the remainder of the procedure without the user having to hold it in position. Thus, when the expandable portion 302 is in an expanded state, the anal canal tissue is positioned between the expandable portion 302 and the proximal element 306. As previously described, the expandable portion 302 and proximal element 306 work together to secure the hub 300 throughout the procedure, such as during vibrational and/or swirling motion of the distal element 402, injection of irrigation fluid, and aspiration of irrigation fluids and fecal matter, etc. The expandable portion 302 further prevents fecal matter FM from leaking out of the rectum R to maintain a clean procedure.

With the hub 300 in place, the user can optionally inject a fluid through the device 100 into the rectum R. In some embodiments, the fluid can be configured to soften the impacted fecal matter FM to facilitate removal of the fecal matter. Examples of fluids include water, saline, mineral oil, water mixed with soap, a water-based solution with stool softener, and other fluids. The fluid can be delivered, for example, through the second channel and second port. Additionally or alternatively, the fluid can be delivered through the first channel and opening. Either way, the fluid passes from an extracorporeal location (such as a syringe or other fluid source) through the hub 300 and into the rectum R. Infusion of one or more fluids can occur before, during, and/or after actuation of the distal element 402, or in some cases not at all. Likewise, infusion of one or more fluids can occur before, during, and/or after aspiration, and aspiration can occur before, during and/or after actuation of the distal element 402, or in some cases not at all.

The distal element 402 can be moved in a circular and/or swirling motion (via the energy source, manually, or both) within the rectum R to disrupt or otherwise break up the fecal matter into smaller and/or softer pieces, and/or induce rectal muscle constriction. Vibratory motion may be applied to the distal element 402 before, during, and/or after the whirling motion. Optionally, aspiration and/or infusion of fluids can occur while the distal element 402 is activated and breaking up fecal matter to remove the treated fecal matter from the treatment site.

At the conclusion of the procedure, when all the stool particles and liquid stool slurry have been removed, the user can collapse the expandable portion 302 of the hub 300 and pull the elongate member 404 proximally to withdraw the distal element 402 and hub 300 from the rectum R.

The entire device 100 may be disposable, or in some cases only the patient-contacting portions of the device 100 (such as the distal element 402 and hub 300) are disposable while the energy source and motor can be reusable. In such embodiments, the distal element 402 and hub 300 can be detachable from the elongate member 404 and handle 406, or the distal element 402, hub 300, and elongate member 404 are detachable from the handle 406.

### CONCLUSION

Although many of the embodiments are described above with respect to systems, devices, and methods for breaking up and/or removing fecal matter from the rectum, the technology is applicable to other applications and/or other approaches, such as Hirschsprung's disease, pelvic floor dyssynergia, neurodegenerative conditions (i.e., Parkinson's disease, multiple sclerosis, spinal cord injury, etc.) associated with chronic recurrent episodes of OC and FI. The devices of the present technology can be used in an at-home or other clinical settings, and may be size-adjusted for use with adult and pediatric patients. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-21.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A device for removing fecal matter from an intestine of a patient, the device comprising:
a proximal portion configured to be engaged by a user and a distal portion configured to be positioned at a treatment site within a lumen of the intestine proximate to impacted fecal matter;
a disruptor comprising a proximal region configured to be handled by a user, a distal region, and an elongated member extending therebetween, wherein the disruptor further comprises:
a distal element carried by a distal portion of the elongate member and configured for insertion into the intestinal lumen through an anus of the patient,
an energy source operably coupled to the distal element, and
wherein activation of the energy source energizes the distal element, thereby causing the distal element to deliver energy to the impacted fecal matter and dislodge a portion of the impacted fecal matter;
an expandable member configured to be positioned in the intestinal lumen, the expandable member having a collapsed state for introduction into the intestinal lumen and an expanded state in which the expandable member is configured to conform to the inner wall of the intestinal lumen to prevent or reduce leakage of intestinal fluids and/or fecal matter through the anus to an extracorporeal location; and
a channel extending distally from a port at the proximal portion of the device to an opening at the distal portion of the device, wherein the port is configured to be fluidly coupled to a negative pressure source to apply a negative pressure at the treatment site to remove the portion of the fecal matter and/or other matter present in the intestinal lumen.

2. The device of claim 1, wherein the channel is a first channel, the port is a first port, and the opening is a first opening, and wherein the device further comprises a second channel extending distally from a second port at the proximal portion of the device to a second opening at the distal portion of the device, wherein the second port is configured to be fluidly coupled to a fluid source to deliver a fluid through the second channel to the treatment site.

3. The device of claim 1, wherein the channel is a first channel, the port is a first port, and the opening is a first opening, and wherein the device further comprises a third channel extending distally from a third port at the proximal portion of the device to a third opening at the expandable member, wherein the third port is configured to be fluidly coupled to a positive pressure source to transform the expandable member from a collapsed state to an expanded state.

4. The device of claim 1, wherein the expandable member is a balloon.

5. The device of claim 1, wherein the distal portion is configured for insertion into the colon in an uncovered state.

6. The device of claim 1, wherein the device has a collapsed state for delivery through the anus, and wherein the device in the collapsed state has a cross-sectional dimension of at least 0.5 inches.

7. The device of claim 1, further comprising a proximal element configured to be positioned proximal of the anus at an extracorporeal location, wherein the proximal element has a diameter that is greater than a diameter of the anus such that the expandable member and the proximal element together secure the device at the anus.

8. The device of claim 1, wherein the energy source is a motor, and wherein activation of the motor causes the distal element to move, thereby dislodging a portion of the fecal matter.

9. The device of claim 8, wherein activation of the motor causes the distal element to vibrate, translate, and/or rotate.

10. The device of claim 1, wherein the energy source is configured to deliver high frequency ultrasound to the impacted fecal matter via the distal element.

11. The device of claim 1, further comprising a hub, wherein the expandable member is a first portion of the hub and the hub further comprises a second, non-expandable portion.

12. The device of claim 11, wherein the first portion of the hub is configured to be positioned in a rectum of the patient and the second portion of the hub is configured to be positioned within an anal canal of the patient.

13. A device for removing fecal matter from an intestine of a patient, the device comprising:
a proximal portion configured to be engaged by a user and a distal portion configured to be positioned at a treatment site within a lumen of the intestine proximate to impacted fecal matter;
a disruptor comprising a proximal region configured to be handled by a user, a distal region, and an elongate member extending therebetween, wherein the disruptor further comprises a distal element carried by a distal portion of the elongate member and configured for insertion into the intestinal lumen through an anus of the patient, wherein the distal element is actuatable to dislodge a portion of the impacted fecal matter; and
a hub having a distal portion configured to be positioned in the intestinal lumen, a proximal portion configured to be positioned proximal of the anus at an extracorporeal location, and an intermediate portion extending between the distal and proximal portions of the hub that is configured to be positioned along the anal canal, wherein the hub further comprises a channel that is configured to slidably receive the elongate member of the disruptor therethrough.

14. The device of claim 13, wherein the distal portion of the hub comprises an expandable member having a collapsed state for introduction into the intestinal lumen and an expanded state in which the expandable member is configured to conform to the inner wall of the intestinal lumen to prevent or reduce leakage of intestinal fluids and/or fecal matter through the anus to an extracorporeal location.

15. The device of claim 14, wherein the expandable member is configured to be deployed in the rectum, adjacent a distal end of the anal canal.

16. The device of claim 14, wherein the proximal portion of the hub has an axial cross-sectional dimension greater than a diameter of the anus such that the proximal portion of the hub cannot move distally through the anus.

17. The device of claim 16, wherein the proximal portion of the hub has a substantially fixed cross-sectional dimension.

18. The device of claim 16, wherein the proximal portion of the hub has a collapsed state and an expanded state in which the hub has the axial cross-sectional dimension greater than the diameter of the anus.

19. The device of claim 14, wherein the channel is a first channel and the hub further comprises a second channel extending distally from a proximal opening at the proximal portion of the hub to a distal opening at the distal portion of the hub, wherein the second channel is configured to be fluidly coupled to a negative pressure source to apply a negative pressure at the treatment site to remove the portion of the fecal matter and/or other matter present in the intestinal lumen.

20. The device of claim 19, wherein the second channel is configured to be fluidly coupled to a fluid source and deliver a fluid to the treatment site.

21. The device of claim 19, wherein the hub further comprises a third channel extending distally from a proximal opening at the proximal portion of the hub to a distal opening at the distal portion of the hub, wherein the third channel is configured to be fluidly coupled to a fluid source and deliver a fluid to the treatment site.

22. The device of claim 14, wherein the hub comprises a sidewall defining an interior region of the hub that is configured to receive all or a portion of the distal element therein.

23. The device of claim 14, wherein the hub has a collapsed configuration and an expanded configuration, and wherein, at least in the collapsed configuration, the hub has a tapered distal region.
